(19) United States
(12) Patent Application Publication
(10) Pub. No.: US 2011/0275872 A1
(43) Pub. Date: Nov. 10, 2011

(54) PROCESS FOR THE SYNTHESIS OF ETHYNYLCYCLOPROPANE

(76) Inventors: Paul Hanselmann, Brig-Glis (CH); Heilam Wong, Guangdong (CN); Yanlei Chen, Foshan Guangdong (CN)

(21) Appl. No.: 12/922,369

(22) PCT Filed: Mar. 26, 2009
(86) PCT No.: PCT/EP2009/002206

§ 371 Date: Nov. 30, 2010

Related U.S. Application Data
(60) Provisional application No. 61/064,788, filed on Mar. 26, 2008.

(30) Foreign Application Priority Data
Mar. 26, 2008 (EP) 08005556.9

(51) Int. Cl. C07C 1/20

(52) U.S. Cl. 585/357

(57) ABSTRACT

Ethynylcyclopropane is prepared in a two-step process by reacting (1,1-dimethoxyethyl)-cyclopropane with a thiol and eliminating the thiol from the intermediate.

The present invention relates to a two step process for the preparation of ethynylcyclopropane of the formula

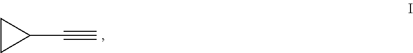

which is an intermediate in the synthesis of pharmaceutical active ingredients, for example of antiviral agents like (−)-6-chloro-4-cyclopropylethynyl-4-trifluoromethyl-1,4-dihydro-2H-3,1-benzoxazin-2-one, a potent HIV reverse transcriptase inhibitor (WO-A 96/22955).

The synthesis of the compound of formula I as disclosed in U.S. Pat. No. 6,552,239 starts from cyclopropanecarboxaldehyde and a diazophosphonate. This reaction is also known as Seyferth-Gilbert alkyne synthesis. Cyclopropanecarboxaldehyde is obtained from (1,1-di-chloroethyl)cyclopropane by eliminating two molecules of hydrogen chloride. A disadvantage of this process is that the preparation of the geminal dichloride requires drastic conditions (e.g. PCl₅) and that at least 2 equivalents of halide waste per equivalent of product have to be disposed.

EP-A-0936207 and GB-A-2355724 both disclose a cyclopropane ring-closure strategy. A 1,3-dihalopropane is reacted with an acetylide derivative and the resulting pentyne is cyclized in the presence of an organometal compound, for example n-butyllithium. The compound of formula I then is obtained in one or two further steps. These processes starting from 1,3-dihalopropane also produce at least two equivalents of halide waste per equivalent of product. A further disadvantage in an industrial scale is the very low reaction temperature of about −70 to −100° C.

In another process as disclosed in EP-A 922686 two equivalents of methanol are directly eliminated of (1,1-dimethoxyethenyl)cyclopropane.

U.S. Pat. No. 6,297,410 discloses another approach wherein during the elimination enyne by-products are generated which are extremely difficult to separate from the product.

The process of U.S. Pat. No. 6,072,094 involves an elimination step of a cis- and trans-vinyl thioether mixture. The preparation of said vinyl thioether requires a silylating agent. Depending on the cis/trans-balance of the starting material the product has to be separated from two different starting compounds. Since the final step is carried out with a cis/trans-isomeric mixture it involves both a cis- and a trans-elimination. However, the optimum reaction conditions for a cis-elimination are different from that of a trans-elimination.

The object of the present invention was thus to provide an alternative process for the preparation of ethynylcyclopropane which does not require very low temperatures and produces little or even no halide waste. Finally a process should be established wherein the final elimination step is carried out starting from a single compound and not from an isomeric mixture.

According to the invention, these objects are achieved by the following process.

Claimed is a process for the preparation of ethynylcyclopropane of the formula

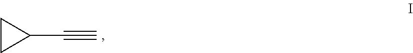

wherein in a first step (1,1-dimethoxyethyl)cyclopropane of the formula

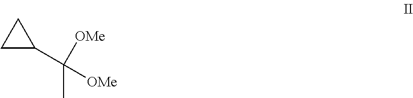

is reacted with a thiol of formula

HSR¹ III,

wherein R¹ is selected from the group consisting of C₁-C₆ alkyl, C₃-C₆ cycloalkyl, aryl and aralkyl wherein each alkyl, cycloalkyl, aryl and aralkyl can carry one or more halogen atoms, nitro, amino groups and/or a further substituent selected from the group consisting of C₁-C₆ alkyl C₁-C₆ alkoxy, and C₃-C₆ cycloalkyl, wherein such further substituent is optionally substituted with one or more halogen atoms, while two equivalents of methanol per equivalent of compound of formula II are eliminated, and a compound of formula

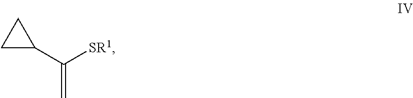

wherein R1 is as defined above, is obtained,
of which, in the second step, the thiol of formula III is eliminated to give the compound of formula I.

An advantage of the present process over the state of the art is that no halide waste is generated in the reaction.

Another advantage is that the second step of the present process can be carried out without heating the reaction mixture. Therefore the danger of formation of undesired by-products is reduced. Also, the instant process provides more than doubled overall yields (calculated on (1,1-dimethoxyethyl)-cyclopropane) as compared to EP-A 922686.

A further advantage is that the elimination step starts from a single starting compound. Therefore, the problem of isomeric mixtures is circumvented. Furthermore, the invention provides an efficient synthesis of ethynylcyclopropane with the use of inexpensive reagents.

Unlike the process disclosed in U.S. Pat. No. 6,072,094 where a mixture of E- and Z-isomers of (2-cyclopropylvinyl)sulfanyl-benzene eliminates one equivalent of thiophenol to give ethynylcyclopropane in a moderate yield, the present (1-cyclopropylvinyl)sulfanyl-benzene provides a more sterically accessible proton and involves no cis- and trans-elimination. Therefore it gives a better yield.

The term “C₁-Cₙ alkyl”, for example “C₁-C₁₈ alkyl”, represents a linear or branched alkyl group having 1 to n carbon atoms. C₁-C₁₈ alkyl represents for example methyl, ethyl, propyl, isopropyl, butyl, isobutyl, sec-butyl, tert-butyl, pentyl, hexyl, octyl, decyl, dodecyl and octadecyl.

The term “C₁-Cₙ alkoxy”, for example “C₁-C₆ alkoxy”, represents a linear or branched alkoxy group having 1 to n carbon atoms. C₁-C₆ alkoxy represents for example methoxy, ethoxy, propoxy, isopropoxy, n-butoxy, isobutoxy, sec-butoxy, tert-butoxy, pentyloxy and hexyloxy.

The term “C₃-Cₙ cycloalkyl”, for example “C₃-C₁₀ cycloalkyl”, represents a cycloaliphatic group having 3 to n carbon atoms. C₃-C₁₀ cycloalkyl represents for example mono- and polycyclic ring systems such as cyclopropyl, cyclobutyl, cyclopentyl, cyclohexyl, cyclo-heptyl, cyclooctyl, adamantyl or norbornyl.

The term aryl represents an aromatic group, optionally substituted with one or more halogen atoms, amino groups, and/or optionally substituted C₁-C₆ alkyl, C₁-C₆ alkoxy or di-C₁-C₆ alkylamino groups. C₆-C₂₀ Aryl represents for example phenyl, naphthyl and derivatives thereof as outlined above.

The term aralkyl represents an aromatic group, wherein the alkyl moiety of the aralkyl residue is linear C₁-C₈ alkyl and the aryl moiety is selected from the group consisting of phenyl, naphthyl, furanyl, thienyl, benzo[b]furanyl, benzo[b]thienyl, optionally being substituted with one or more halogen atoms, amino groups, and/or optionally substituted C₁-C₆ alkyl, C₁-C₆ alkoxy or di-C₁-C₆ alkylamino groups.

Preferably, the first step of said process is carried out in the presence of at least one acidic catalyst. Furthermore, the acidic catalyst can be a solid acid or added as a salt like iron(III) chloride. Further preferred, said catalyst is a non-volatile inorganic or organic acid. Said organic acid can also be present in the form of its anhydride.

Suitable inorganic acids herein, include sulfuric acid, sulfurous acid, phosphoric acid, phosphorous acid, nitric acid, hydrobromic acid, hydrochloric acid, perchloric acid and boric acid, as well as Lewis acids such as include tin(II) chloride, tin(IV) chloride, tin(II) bromide, tin(IV) bromide, boron trifluoride, boron trifluoride etherate, trialkylaluminum, dialkyl-aluminum chlorides, alkylaluminum dichlorides, aluminum chloride, boron trichloride, boron tribromide, scandium bromide, scandium chloride, scandium iodide, titanium chloride, titanium bromide, iron(III) chloride, vanadium chlorides, chromium chlorides, manganese chlorides, zirconium chloride, zirconium bromide, zinc bromide, zinc chloride, yttrium chlorides, molybdenum chlorides, ruthenium chlorides and lanthanide chlorides such as lanthanum, cerium or ytterbium trichloride.

Suitable organic acids include alkanesulfonic acids such as methanesulfonic acid, butanesulfonic acid, (+, − or ±)-10-camphorsulfonic acid and arenesulfonic acids such as aminobenzene-2-sulfonic acid, 8-amino-naphthalene-1-sulfonic acid, benzenesulfonic acid, 2,5-diaminobenzenesulfonic acid, 5,6-bis(dimethylamino)-1-naphthalenesulfonic acid, 4-hydroxy-2-naphthalenesulfonic acid, 4-hydroxy-3-nitroso-1-naphthalenesulfonic acid tetrahydrate, 8-hydroxyquinoline-5-sulfonic acid, 1-naphthalenesulfonic acid, 2-naphtha-lenesulfonic acid, 1,5-naphthalenedisulfonic acid, 2,6-naphthalenedisulfonic acid, 2,7-napththalenedisulfonic acid, phenylboric acid, 2,4,6-trinitrobenzenesulfonic acid hydrate, 2-(2-pyridyl)ethanesulfonic acid, 2-(4-pyridyl)ethanesulfonic acid, 3-pyridine-sulfonic acid, (2-hydroxypyridyl)methanesulfonic acid, sulfanilic acid, 2-sulfobenzoic acid hydrate, 5-sulfosalicylic acid hydrate, p-toluenesulfonic acid or 2,4-xylenesulfonic acid, as well as organic Lewis acids such as scandium trifluoromethansulfonate, silver trifluoro-methanesulfonate, copper(I) trifluoromethanesulfonate and copper(II) trifluoromethane-sulfonate.

An inorganic or organic solid acid can also be used as acidic catalyst. Suitable organic solid acidic catalysts include acidic ion exchange resins having sulfonic acid groups such as Amberlyst® or Amberlite®.

Suitable inorganic solid acidic catalysts can be selected from the group consisting of acidic aluminum oxides, acidic aluminosilicates and/or acidic silicates such as acidic zeolithes or montmorillonites, each suitable for heterogeneous catalysis.

The second step is preferably carried out in the presence of at least one equivalent of a strong base, preferably of at least two equivalents, more preferably of 2.5 to at 3 equivalents. Under basic conditions, ethynylcyclopropane most probably is initially produced in the deprotonated form as an acetylide, which, during aqueous workup, is protonated again to give the final product of formula I.

Suitable strong bases are organic “super bases” or alkali metal bases.

Organic super bases mean nitrogen containing polycyclic compounds having high proton affinities and basicities and contain extended π-systems, such as 1,5-diazabicycle[4.3.0]-non-5-ene (DBN) or 1,8-diaza-bicyclo[5.4.0]undec-7-ene (DBU).

Strong alkali metal bases in the meaning of the present process are for example alkali metal alkylides, wherein optionally the alkyl moiety has further substitutents; alkali metal C₁-C₆ alkylamides, wherein optionally the alkyl moiety has further substitutents; aryl alkali metal compounds, wherein optionally the aryl moiety has further substitutents; or alkali metal silazides, wherein optionally the silyl moiety has further substitutents. Examples of suitable alkali metal bases are methyllithium, ethyllithium, propyllithium, isopropyllithium, n-butyllithium, isobutyllithium, tert-butyllithium, pentyllithium, hexyllithium, lithium tri-phenylmethylide, phenyllithium, tolyllithium, lithium diisopropylamide (LDA), potassium diisopropylamide (KDA), sodium diisopropylamide (NDA), lithium 3-(aminopropyl)amide (LAPA), potassium 3-(aminopropyl)amide (KAPA), sodium 3-(aminopropyl)amide (NAPA), potassium bis(trimethylsilyl)-amide, lithium hexamethyldisilazide (LiHMDS), potassium hexamethyldisilazide (KHMDS) and sodium hexamethyldisilazide (NaHMDS).

Particularly preferred the strong base is selected from the group consisting of KAPA, LAPA, NAPA, LDA, KDA, NDA, LiHMDS, KHMDS and NaHMDS.

Most preferably the strong base is selected from the group consisting of KAPA, LAPA and NAPA.

The examples below illustrate how the process according to the invention is carried out, without limiting it thereto.

### EXAMPLES

### Example 1

### (1,1-Dimethoxy-ethyl)-cyclopropane (II)

To a flask charged with cyclopropyl methyl ketone (50 g, 0.59 mol) was added trimethyl orthoformate (82 g, 0.77 mol), methanol (200 mL, 4.94 mol) and p-toluenesulfonic acid (0.25 g, 1.45 mmol) at 25° C. respectively. The solution was stirred at 25° C. for 2 h. Sodium methoxide (0.15 g, 2.78 mmol) were added to the solution under vigorous stirring. Methyl formate, methanol and trimethyl orthoformate were removed under reduced pressure (at about 250-300 mbar). The remainder was washed with brine (100 mL) and dried over Na₂SO₄ (15 g). (1,1-Dimethoxy-ethyl)-cyclopropane was obtained as a colorless liquid in 45-65%.

### Example 2

### (1-Cyclopropylvinylsulfanyl)-benzene (IV)

To a flask fitted with an additional funnel was added (1,1-dimethoxy-ethyl)-cyclopropane (13 g, 0.10 mol), thiophenol (11 g, 0.10 mol), toluene (250 mL) and 10-camphorsulfonic acid (700 g, 3 mmol) respectively. A pressure-equalizing dropping funnel was charged with molecular sieve (beads) and fitted with a reflux condenser on top. The solution was refluxed at 130° C. (bath temperature) for 20 h. The mixture was washed with 10% NaOH aqueous solution (2×50 mL) and brine (100 mL) and dried over Na₂SO₄ (5 g). Toluene was removed under reduced pressure to give an oil containing 46% of 1-cyclopropylvinylsulfanyl)-benzene. The raw product can be further purified, for example by distillation.

### Example 3

### Ethynylcyclopropane (I)

KAPA (potassium aminopropyl amide) was generated in situ either by potassium hydride and 1,3-diaminopropane (Brown, C. A. J., Org. Chem. 1978, 43, 3083-3084) or by potassium and 1,3-diaminopropane in the presence of a catalyst Fe(NO₃)₃ (Kimmel, T. et al. Org. Chem. 1984, 49, 2494-2496). (1-cyclopropylvinyl)sulfanyl)-benzene (358 mg, 2.03 mmol) was added drop wise to a flask charged with a freshly generated KAPA solution (10 mL, 1.27 M in 1,3-diaminopropane, 12.7 mmol) at 0° C. (bath temperature). The mixture was stirred at 25° C. for 3 h and then cooled to 0° C. To the mixture was added ice water. Yield of ethynylcyclopropane in the raw product is 88%. The mixture was washed with 10% AcOH aqueous solution (2×50 mL) and brine (100 mL) and dried over Na₂SO₄ (2 g). The raw product was purified by distillation. Bp.: 50-51° C.

1. A process for the preparation of ethynylcyclopropane of the formula 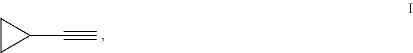
characterized in that in a first step (1,1-dimethoxyethyl)cyclopropane of the formula 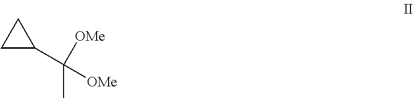
is reacted with a thiol of formula
HSR¹ III,
wherein R¹ is selected from the group consisting of C₁₋₆-alkyl, C₃₋₆-cycloalkyl, aryl and aralkyl wherein each alkyl, cycloalkyl, aryl and aralkyl can carry one or more halogen atoms, nitro, amino groups and/or a further substituent selected from the group consisting of C₁₋₆-alkyl C₁₋₆-alkoxy, and C₃₋₆-cycloalkyl, wherein such further substituent is optionally substituted with one or more halogen atoms, while two equivalents of methanol per equivalent of compound of formula II are eliminated, and a compound of formula 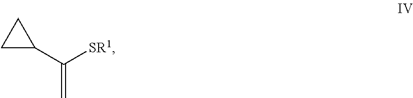
wherein R¹ is as defined above, is obtained,
of which, in the second step, the thiol of formula III is eliminated to give the compound of formula (I).

2. The process of claim 1, characterized in that the first step is carried out in the presence of an acidic catalyst.

3. The process of claim 2, characterized in that the catalyst is an inorganic or organic acid, or an anhydride thereof.

4. The process of claim 1, characterized in that the second step is carried out in the presence of at least two equivalents of a strong base, preferably of at least three equivalents, more preferably of at least six equivalents.

5. The process of claim 4, characterized in that the strong base is an organic super base or an alkali metal base.

6. The process of claim 5, characterized in that the organic super base is 1,5-diaza-bicyclo[4.3.0]non-5-ene (DBN) or 1,8-diaza-bicyclo[5.4.0]undec-7-ene (DBU).

7. The process of claim 5, characterized in that the alkali metal base is selected from the group consisting of optionally substituted alkali metal alkylides, optionally substituted alkali metal (C₁-C₆)alkylamides, aryl alkali metal compounds and alkali metal silazides.

8. The process of claim 5, characterized in that the alkali metal base is selected from the group consisting of methyllithium, ethyllithium, propyllithium, isopropyllithium, w-butyllithium, isobutyllithium, tert-butyllithium, pentyllithium, hexyllithium, lithium triphenylmethylide, phenyllithium, tolyllithium, lithium diisopropylamide (LDA), potassium diisopropylamide (KDA), sodium diisopropylamide (NDA), lithium 3-(aminopropyl)amide (LAPA), potassium 3-(aminopropyl)amide (KAPA), sodium 3-(aminopropyl)amide (NAPA), potassium bis(trimethylsilyl)-amide, lithium hexamethyldisilazide (LiHMDS), potassium hexamethyldisilazide (KHMDS) and sodium hexamethyldisilazide (NaHMDS).
